(19) United States
(12) Patent Application Publication
(10) Pub. No.: US 2012/0217415 A1
(43) Pub. Date: Aug. 30, 2012

(54) Clean-as-a-whistle cleaning system: product specification

(76) Inventor: Stanley Quinn Wormely, Leeds, AL (US)

(21) Appl. No.: 12/929,409

(22) Filed: Feb. 28, 2011

(51) Int. Cl. A61L 2/10

(52) U.S. Cl. 250/455.11

(57) ABSTRACT

The Clean as a Whistle Cleaning System is a product that will sanitize and deodorize whistles without the use of a liquid. The product accomplishes this goal by the use of a unique ultraviolet bulb. The bulb will be powered by AA batteries which will work in conjunction with the circuit board. The compact and very portable units will be equipped with a power button that will start the cleaning process and automatically shut off the units after the cleaning process has been completed. The process will take no longer than 15 minutes to complete. In addition, the units will also be designed to store additional sanitized whistles.

### DISCLOSURE OF THE ENTIRE ARTICLE

### Specification

I, Stanley Q. Wormely have invented a new design for a whistle cleaning system as set forth in the following specification:

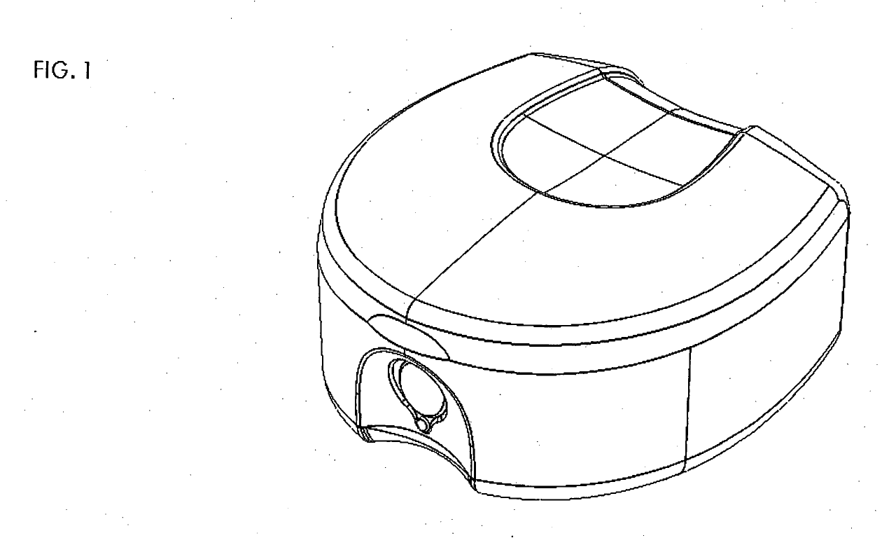

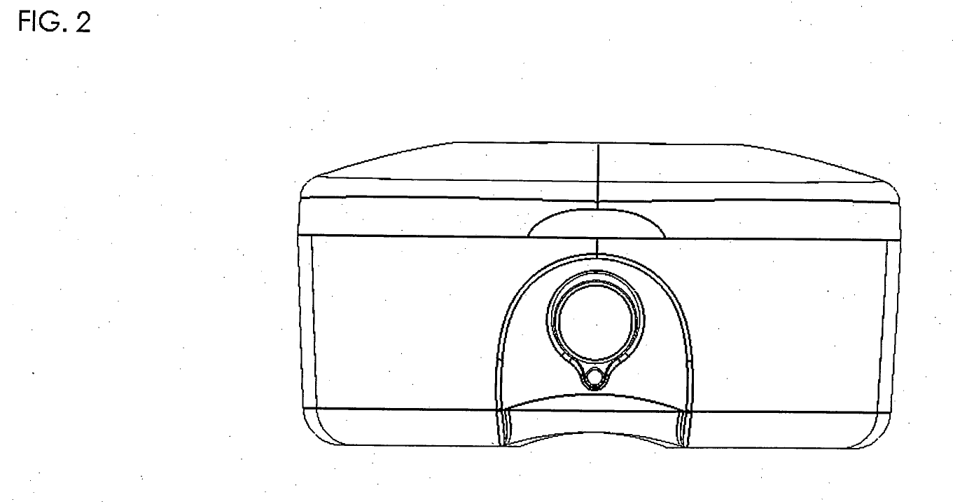

FIG. 1 is a top elevational view of cleaning system showing my new design;

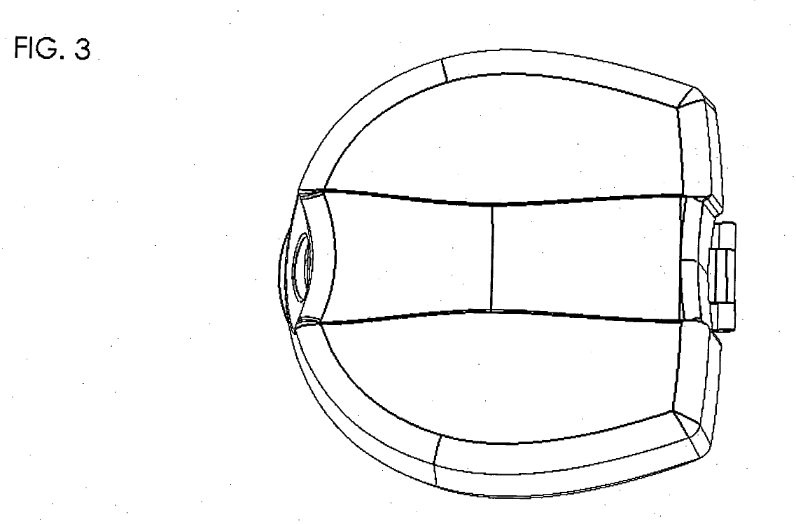

FIG. 2 is a front plan view thereof;

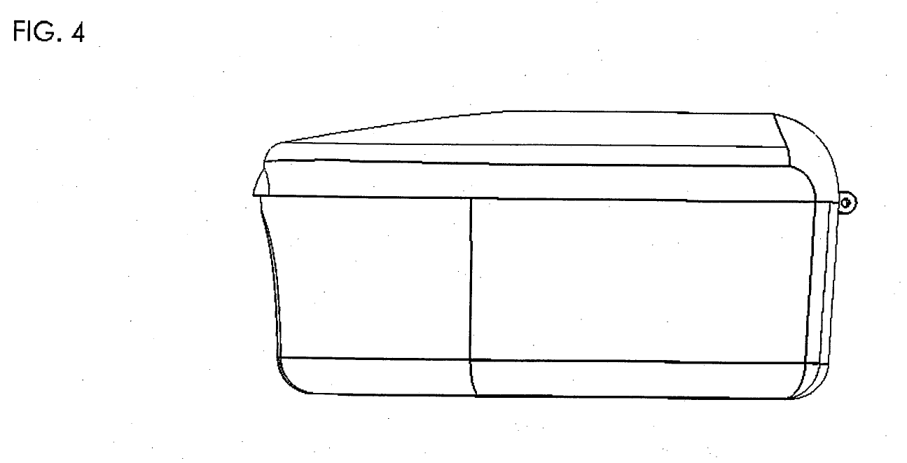

FIG. 3 is a bottom elevational view thereof;

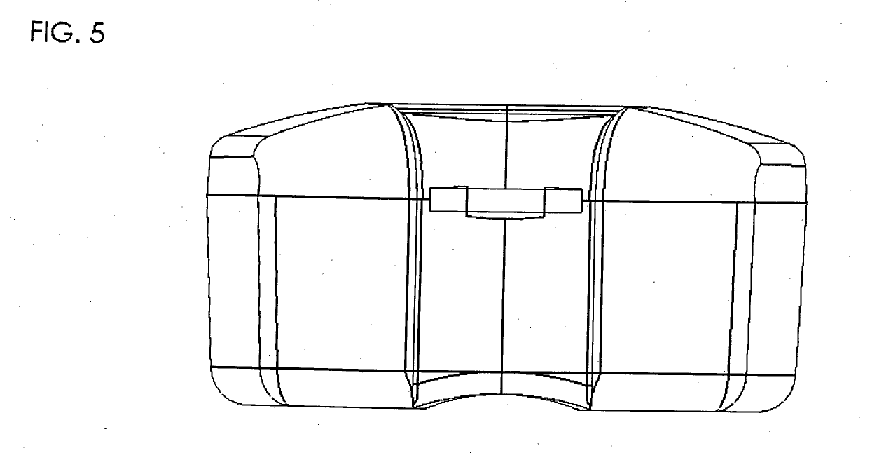

FIG. 4 is a right side plan view thereof;

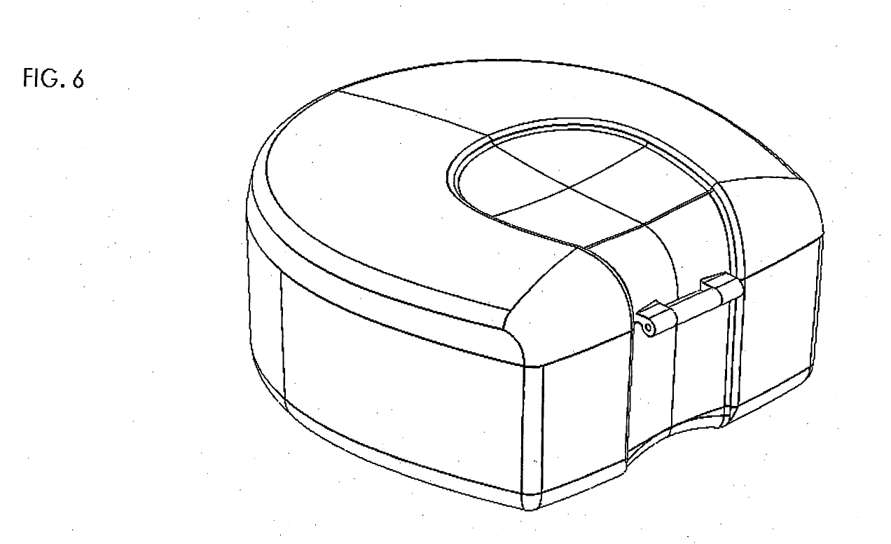

FIG. 5 is a rear side plan view thereof;

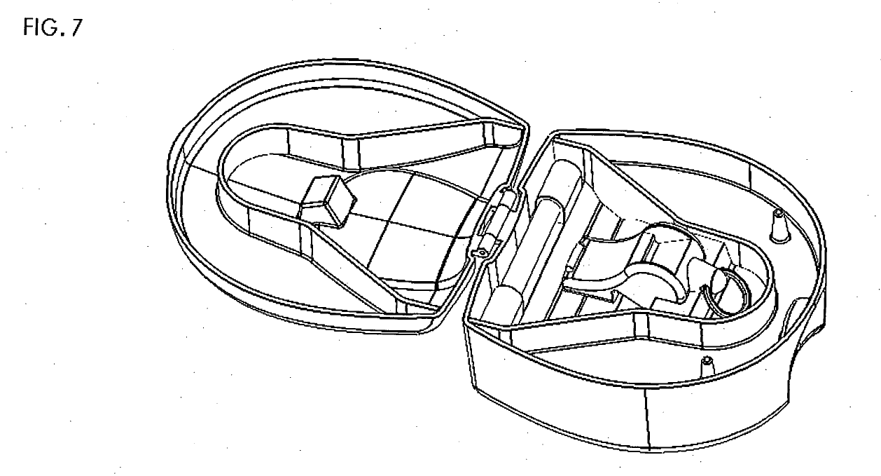

FIG. 6 is a perspective top elevational view thereof;

The color shown on the claimed design forms no part thereof. The left side plan view of the design is a mirror image of the right side.

Drawing Disclosure

1. I claim: the ornamental design for a whistle cleaning system as shown and described.
